(19) United States
(12) Patent Application Publication
(10) Pub. No.: US 2026/0130864 A1
(43) Pub. Date: May 14, 2026

(54) METHOD AND COMPOSITION FOR ENHANCING EXERCISE PERFORMANCE

(71) Applicant: NANJING NUTRABUILDING BIO-TECH CO., LTD., Nanjing (CN)

(72) Inventors: Ronghua YI, Nanjing (CN); Kylin LIAO, Nanjing (CN); Shawn WELLS, Frisco, TX (US)

(21) Appl. No.: 19/443,933

(22) Filed: Jan. 8, 2026

Related U.S. Application Data
(63) Continuation of application No. PCT/CN2024/104348, filed on Jul. 9, 2024.

(30) Foreign Application Priority Data
Jul. 12, 2023 (WO) PCT/CN2023/107034

(51) Int. Cl. A61K 31/121; A23K 20/111; A61P 1/00; A61P 25/00; A61P 43/00

(52) U.S. Cl. CPC A61K 31/121; A23K 20/111; A61P 1/00; A61P 25/00; A61P 43/00

(57) ABSTRACT

The invention discloses a method and composition for regulating or increasing intestinal flora, and applications thereof in preparing nutritional products, health care products, foods and beverages for regulating intestinal flora. The composition comprising dehydrozingerone of the invention can significantly regulate or increase the level of intestinal flora in a subject, increase dopamine in brain by regulating or increasing the level of intestinal flora in the subject, thereby enhancing exercise performance.

### CROSS REFERENCE TO RELATED APPLICATION

This application is a continuation application of International Patent Application No. PCT/CN 2024/104348, filed on Jul. 9, 2024, which claims the priority of the International Application No. PCT/CN 2023/107034, filed on Jul. 12, 2023, the contents of all of which are incorporated herein by reference in their entirety.

### FIELD OF THE INVENTION

The invention belongs to the technical field of health-care foods and dietary supplements, and specifically relates to composition and method for enhancing exercise performance and related applications thereof in nutritional supplements, health-care products, foods and beverages.

### BACKGROUND OF THE INVENTION

Studies have shown that giving broad-spectrum antibiotics to mice to remove their intestinal bacteria will reduce their running performance by about half, that is to say, the difference in running performance is mainly due to the existence of certain intestinal bacteria species in mice with good performance. In addition, it was found that two bacterial species, Eubacterium rectale and Coprococcus eutactus, which are closely related to better running performance, will produce metabolites called fatty acid amides (FAA). Fatty acid amides stimulate Cannabinoid 1 (CB1 ) receptor on the surface of sensory nerves buried in the intestine, and these sensory nerves are connected to the brain through the spine. During exercise, stimulating these CB1 receptor-covered sensory nerves leads to an increase in the level of neurotransmitter dopamine in the brain region of ventral striatum. The ventral striatum is a key node in the brain reward and motivation network.

Dehydrozingerone (DHZ), also known as feruloylmethane, is separated from Curcuma longa and is a structural analogue of curcumin. In vitro antibacterial tests related to food preservation show that DHZ has antibacterial ability and anti-inflammatory effect. Therefore, it is very important to study the relationship between DHZ and intestinal flora, as well as the relationship between DHZ and exercise performance, and then find a suitable nutrition and health care strategy.

### SUMMARY OF THE INVENTION

To achieve the above objectives, the present invention provides the following:

One aspect of the present invention relates to a method for regulating or increasing the level of intestinal flora in a subject, the method includes: administering to the subject a composition comprising dehydrozingerone or a salt, acid, ester or derivative thereof; the intestinal flora is Gram-positive bacteria.

In some embodiments, the Gram-positive bacteria are Eubacterium rectale and/or Coprococcus eutactus.

In some embodiments, the method increases dopamine in brain by regulating or increasing the level of intestinal flora in the subject, thereby enhancing exercise performance. The enhancing exercise performance includes improving endurance, raising heart rate, increasing blood flow, enhancing muscle endurance and increasing muscle strength or motivation.

In some embodiments, the composition comprising dehydrozingerone or a salt, acid, ester or derivative thereof is formulated into nutritional supplement, food, beverage or animal feed.

In some embodiments, the composition comprising dehydrozingerone or a salt, acid, ester or derivative thereof is prepared into solid preparation or liquid preparation.

In some embodiments, the composition comprising dehydrozingerone or a salt, acid, ester or derivative thereof is in the form of suppository, tablet, pill, granule, powder, film, capsule, beverage, aerosol, spirit, tincture, tonic, liquid suspension or syrup.

In some embodiments, the composition comprising dehydrozingerone or a salt, acid, ester or derivative thereof is administered to the subject at a dosage of 1-1500 mg.

In some embodiments, the composition is administered at least once per day for 1 week to 7 weeks, 2 weeks to 7 weeks, 3 weeks to 7 weeks, and 4 weeks to 7 weeks.

In some embodiments, the method includes oral administration, intravenous injection, intramuscular injection, intraperitoneal injection, topical drip, external use or sublingual administration, and other administration routes.

In some embodiments, the composition is administered to the subject such that the daily intake of DHZ is between about 1 mg and about 300 mg, or between about 1 mg and about 200 mg, between about 5 mg and about 200 mg, between about 10 mg and about 150 mg, and between about 20 mg and about 100 mg. In some embodiments, the subject is human. In some embodiments, the dopamine level of the subject administered with the composition comprising dehydrozingerone or a salt, acid, ester or derivative thereof is higher than that in the subject without administration.

Another aspect of the present invention relates to use of a composition comprising dehydrozingerone or a salt, acid, ester or derivative thereof in preparing a nutritional supplement, food, beverage or animal feed for regulating or increasing the level of intestinal flora in a subject, the intestinal flora is Gram-positive bacteria.

In some embodiments, the Gram-positive bacteria are Eubacterium rectale and/or Coprococcus eutactus.

In some embodiments, the composition increases dopamine in brain by regulating or increasing the level of intestinal flora in the subject, thereby enhancing exercise performance. The enhancing exercise performance includes improving endurance, raising heart rate, increasing blood flow, enhancing muscle endurance and increasing muscle strength or motivation.

In some embodiments, the composition is administered at a dosage of 1-1500 mg.

In some embodiments, the composition comprising dehydrozingerone or a salt, acid, ester or derivative thereof is in the form of suppository, tablet, pill, granule, powder, film, capsule, beverage, aerosol, spirit, tincture, tonic, liquid suspension or syrup.

In some embodiments, the composition comprising dehydrozingerone or a salt, acid, ester or derivative thereof is prepared into solid preparation or liquid preparation.

Another aspect of the present invention relates to a composition comprising dehydrozingerone or a salt, acid, ester or derivative thereof for regulating or increasing the level of intestinal flora in a subject, the intestinal flora is Gram-positive bacteria.

In some embodiments, the Gram-positive bacteria are Eubacterium rectale and/or Coprococcus eutactus.

In some embodiments, the composition increases dopamine in brain by regulating or increasing the level of intestinal flora in the subject, thereby enhancing exercise performance. The enhancing exercise performance includes improving endurance, raising heart rate, increasing blood flow, enhancing muscle endurance and increasing muscle strength or motivation.

In some embodiments, the composition is administered at a dosage of 1-1500 mg.

In some embodiments, the composition comprising dehydrozingerone or a salt, acid, ester or derivative thereof is formulated into nutritional supplement, food, beverage, animal feed.

Another aspect of the present invention relates to a method for enhancing exercise performance in a subject, the method includes administering to the subject a composition comprising dehydrozingerone or a salt, acid, ester or derivative thereof.

In some embodiments, the method enhances exercise performance in the subject by increasing dopamine in brain.

In some embodiments, the enhancing exercise performance includes improving endurance, raising heart rate, increasing blood flow, enhancing muscle endurance and increasing muscle strength or motivation.

In some embodiments, the composition comprising dehydrozingerone or a salt, acid, ester or derivative thereof is formulated into nutritional supplement, food, beverage or animal feed.

In some embodiments, the composition comprising dehydrozingerone or a salt, acid, ester or derivative thereof is prepared into solid preparation or liquid preparation.

In some embodiments, the composition comprising dehydrozingerone or a salt, acid, ester or derivative thereof is administered in the form of suppository, tablet, pill, granule, powder, film, capsule, beverage, aerosol, spirit, tincture, tonic, liquid suspension, syrup, functional food, beverage or sublingual product.

In some embodiments, the composition comprising dehydrozingerone or a salt, acid, ester or derivative thereof is administered to the subject at a dosage of 1-1500 mg.

In some embodiments, the composition is administered at least once per day for 1 week to 7 weeks, 2 weeks to 7 weeks, 3 weeks to 7 weeks, and 4 weeks to 7 weeks.

In some embodiments, the method includes oral administration, intravenous injection, intramuscular injection, intraperitoneal injection, topical drip, external use or sublingual administration, and other administration routes.

In some embodiments, the composition is administered to the subject such that the daily intake of DHZ is between about 1 mg and about 300 mg, or between about 1 mg and about 200 mg, between about 5 mg and about 200 mg, between about 10 mg and about 150 mg, and between about 20 mg and about 100 mg. In some embodiments, the subject is human. In some embodiments, the dopamine level of the subject administered with the composition comprising dehydrozingerone or a salt, acid, ester or derivative thereof is higher than that in the subject without administration. In some embodiments, the exercise endurance of the subject administered with the composition comprising dehydrozingerone or a salt, acid, ester or derivative thereof is higher than that in the subject without administration.

Another aspect of the present invention relates to use of a composition comprising dehydrozingerone or a salt, acid, ester or derivative thereof in preparing a nutritional supplement, food, beverage or animal feed for enhancing exercise performance in a subject.

In some embodiments, the composition enhances exercise performance in the subject by increasing dopamine in brain. The enhancing exercise performance includes improving endurance, raising heart rate, increasing blood flow, enhancing muscle endurance and increasing muscle strength or motivation.

In some embodiments, the composition is administered at a dosage of 1-1500 mg.

In some embodiments, the composition comprising dehydrozingerone or a salt, acid, ester or derivative thereof is prepared into solid preparation or liquid preparation.

In some embodiments, the composition comprising dehydrozingerone or a salt, acid, ester or derivative thereof is in the form of suppository, tablet, pill, granule, powder, film, capsule, beverage, aerosol, spirit, tincture, tonic, liquid suspension or syrup.

Another aspect of the present invention relates to a composition comprising dehydrozingerone or a salt, acid, ester or derivative thereof, for enhancing exercise performance in a subject.

In some embodiments, the composition enhances exercise performance in the subject by increasing dopamine in brain. The enhancing exercise performance includes improving endurance, raising heart rate, increasing blood flow, enhancing muscle endurance and increasing muscle strength or motivation.

In some embodiments, the composition is administered at a dosage of 1-1500 mg.

In some embodiments, the composition comprising dehydrozingerone or a salt, acid, ester or derivative thereof is formulated into nutritional supplement, food, beverage, animal feed.

In some embodiments, the composition comprising dehydrozingerone or a salt, acid, ester or derivative thereof is prepared into solid preparation or liquid preparation.

In some embodiments, the composition comprising dehydrozingerone or a salt, acid, ester or derivative thereof is in the form of suppository, tablet, pill, granule, powder, film, capsule, beverage, aerosol, spirit, tincture, tonic, liquid suspension or syrup.

The method of administering the composition comprising dehydrozingerone or a salt, acid, ester or derivative thereof according to the present invention, can significantly regulate or increase the level of intestinal flora in the subject, increase dopamine in brain by regulating or increasing the level of intestinal flora in the subject, thereby enhancing exercise performance. The administration of a composition comprising dehydrozingerone or a salt, acid, ester or derivative thereof can significantly enhance exercise performance, including improving endurance, raising heart rate, increasing blood flow, enhancing muscle endurance and increasing muscle strength or motivation, etc.

### BRIEF DESCRIPTION OF THE DRAWINGS

FIG. 1 shows the treadmill exercise endurance of mice in each group.

FIG. 2 shows the dopamine content in the brain of mice in each group.

FIGS. 3A and 3B are stacked bar and heatmap of cecal fecal flora genus levels, respectively.

FIGS. 4A and 4B show the bacterial copy numbers of Eubacterium rectale and Coprococcus eutactus in cecal feces of each group, respectively.

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. To the contrary, the invention is intended to cover alternatives, modifications and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the terms “subject” or “individual” are used interchangeably to refer to any subject to whom the disclosed methods and compositions can be applied or administered. The subject may have a disease or disorder, but the subject does not need to be sick to benefit from the disclosed methods and compositions. Subjects may need to regulate or improve their intestinal and/or overall health, but they may also have overall healthy intestine and wish to maintain or further regulate or improve their intestinal and/or overall health. Therefore, any subject can take the disclosed composition or become a recipient of the disclosed method. As used herein, the term “subject” refers to animals (for example, birds, reptiles and mammals). In some embodiments, the subject can be mammals including non-primates (e.g., camels, donkeys, zebras, cows, horses, cats, dogs, rats and mice) and primates (e.g., monkeys, chimpanzees and humans). In certain embodiments, the subject may be a non-human mammal. In other embodiments, the subject may be a human.

The term “administer”, “administering”, or “administration”, as used in this invention, refers to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

The present invention relates to, regulating or increasing the level of intestinal flora in a subject, increasing dopamine in brain by regulating or increasing the level of intestinal flora in a subject, thereby enhancing exercise performance, by administering to the subject a composition comprising dehydrozingerone or a salt, acid, ester or derivative thereof at a certain dosage.

Depending on the particular dehydrozingerone-containing formulation and form, contemplated methods include an administration of at least 1 mg/day of dehydrozingerone or dehydrozingerone substitute. This amount per day may be administered at once or in multiple doses. Typically, an effective amount of dehydrozingerone or dehydrozingerone derivative in the dehydrozingerone-containing composition to be administered at once or in multiple doses per day is of or between 1 milligram (mg) to 1500 mg. One or more doses may be administered once a day for any period of time. For example, the effective dose may be administered each day for one day, a few days, multiple days, or on a daily basis indefinitely. Dehydrozingerone can be administered for 7 days or more in a cycle, depending on the desired effect. More typically, the amount of dehydrozingerone or dehydrozingerone derivative to be administered each day is 2 mg, 5 mg, 10 mg, 25 mg, 250 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg. More typically, the amount of dehydrozingerone or dehydrozingerone derivative to be administered each day is 1 mg to 1000 mg. Most typically, the amount of dehydrozingerone or dehydrozingerone derivative to be administered each day is 50 mg.

Multiple forms and formulations of the composition comprising dehydrozingerone are contemplated for regulating or increasing the level of intestinal microbiota in a subject. Compositions will be formulated as a nutritional or dietary supplement, (medical) food item, animal feed, in liquid or solid form, comprising the composition comprising dehydrozingerone, and may optionally also comprise a nutritionally or pharmaceutically acceptable carrier. For example, where the composition is in solid form, the compositions may be formulated as a snack bar, yogurt, lozenge, tablet, or capsule, or may be coated onto cereal products, included in baked goods. On the other hand, where the supplement is in liquid form, the compositions may be formulated as a tincture, soft gel capsule, liquid capsule, syrup, carbonated drink, a brewed beverage (e.g., as coffee or tea), a juice, an energy drink, a sports drink, or flavored water. While nutritional and pharmaceutical compositions for human use are especially contemplated, it should be appreciated that the composition and formulation comprising dehydrozingerone may also be employed for veterinary use (e.g., use in animal feed for domestic companion animals (‘pets’) or in animal feed for farm animals. In further contemplated aspects, the composition comprising dehydrozingerone may also be provided as a bulk product (e.g., in quantities of equal or greater than 100 g, equal or greater than 1,000 g, or equal or greater than 10 kg) for use in production of the nutritional supplement, a (medical) food item, animal feed.

### EXAMPLES

Twenty-four pure Swiss male mice were randomly divided into three groups, with 8 mice in each group, and were given food and water freely under the light and dark cycle of 12:12 hours. The first group was the control group (group 1, no DHZ), the second group was given by gavage 50 mg/kg DHZ (group 2), and the third group was given by gavage 100 mg/kg DHZ (group 3). DHZ solution was prepared with 5% DMSO, 25% PEG-400, 30% propylene glycol and 40% normal saline. All the mice were administered at a dose of 10 mL/kg body weight for 4 weeks. 4 weeks later, these mice were made to run on treadmill voluntarily for exercise analysis, and the running distance of each of mice when exhausted was recorded. All the mice were untrained before exercise. Each group was perfused with paraformaldehyde, and brain tissue was frozen in liquid nitrogen for ELISA, and then dopamine was detected. The cecal feces of mice were collected and the flora was sequenced by 16s. The proportion of different bacteria in feces was obtained, and the DNA in feces was extracted to detect the copy number of Eubacterium rectale and Coprococcus eutactus. The statistical analysis of data was conducted by unpaired t-test, and * indicates significant difference. Compared with the control group, * means p<0.05; ** means p<0.01; *** means p<0.001; **** means p<0.0001.

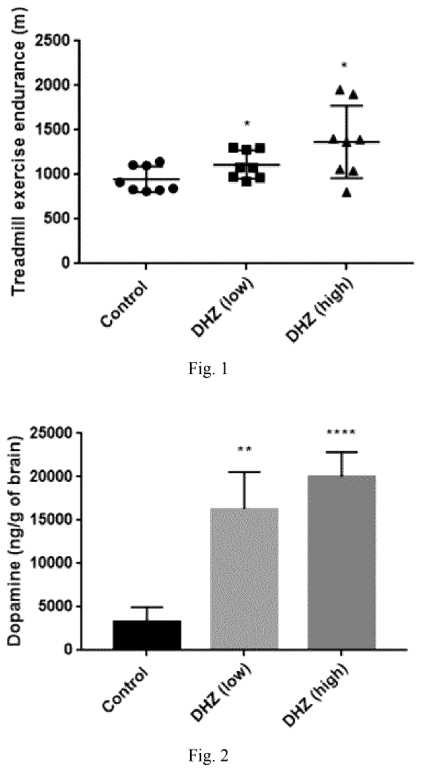

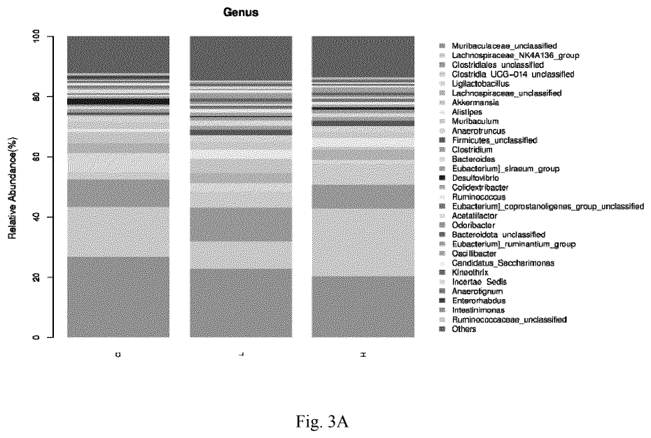

FIG. 1 shows the treadmill exercise endurance of mice in each group after administration for 4 weeks. Compared with the control group (948 m), DHZ low-dose group (1112 m) and high-dose group (1367 m) significantly improve exercise endurance. The exercise performance of DHZ high-dose group is 44% higher than that of control group. FIG. 2 shows the dopamine content in the brain of mice in each group after administration for 4 weeks. The level of dopamine in DHZ groups is higher than that in the control group, with 16,285 ng/g brain in the low dose group and 19,988 ng/g brain in the high dose group, which is 5 times higher than that in the control group (3,293 ng/g brain). Therefore, we can conclude that DHZ supplementation may enhance or improve exercise or sports performance by increasing brain dopamine level.

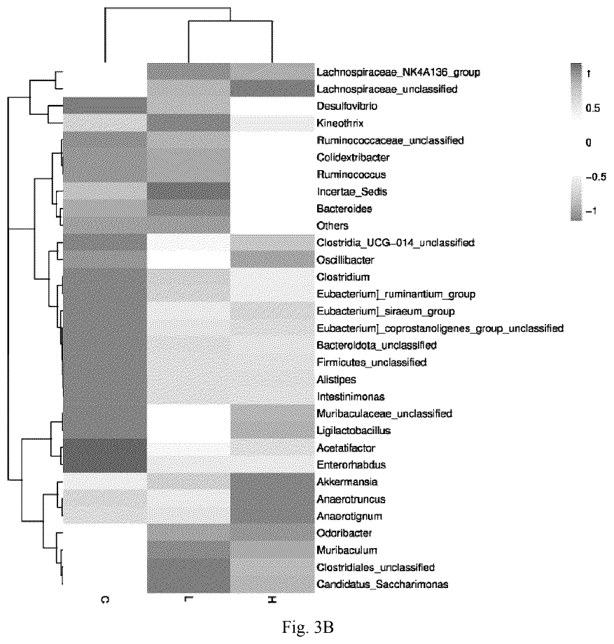

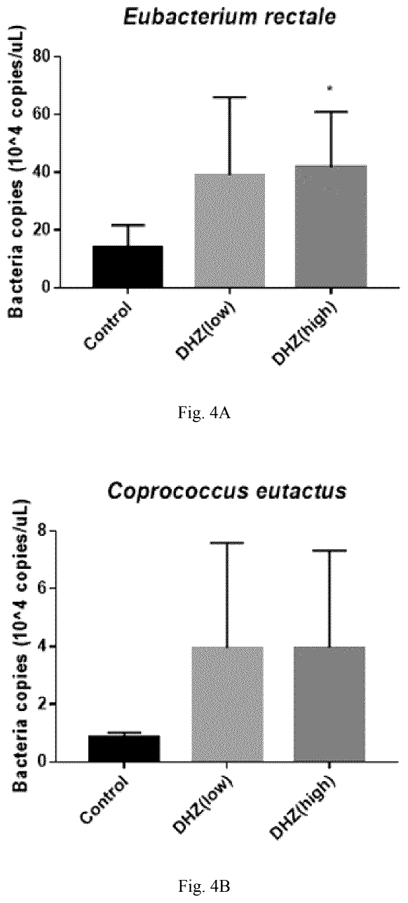

FIGS. 3A and 3B are stacked bar and heatmap of cecal fecal flora genus levels in each group after administration for 4 weeks (C control; L low dose group; H high dose group), respectively. Stacked bar shows the composition of highly expressed species in each group, and can observe the composition and expression of species within the group and the trend of species expression between groups. The heatmap uses the gradient from blue to red to reflect the change of abundance from low to high. The closer to blue (−1), the lower the abundance, and the closer to red (1), the higher the abundance. The results show that after administrating DHZ for 4 weeks, the number of Ruminococcaceae unclassified, Colidextribacter, Ruminococcus, Incertae Sedis Bacteroides, Clostridia UCG-014 unclassified, Oscillibacter, Clostridium, Eubacterium ruminantium group, Eubacterium siraeum group, Eubacterium coprostanoligenes group unclassified, Bacteroidota unclassified, Firmicutes unclassified, Alistipes and Intestimonas in the intestine increases compared with the control group. Among them, the bacteria related to exercise—Eubacteria increase. FIGS. 4A and 4B show the bacterial copy numbers of Eubacterium rectale and Coprococcus eutactus in cecal feces of each group, respectively. The more copy numbers, the more bacterial colonization. The results show that after 4 weeks of DHZ administration, the number of Eubacterium rectale and Coprococcus eutactus increases by about 3 times compared with the control group. Therefore, it is found that after taking DHZ for 4 weeks, the intestinal flora changes. The abundance of Eubacterium rectale and Coprococcus eutactus has increased. Specific intestinal flora regulates the brain-gut axis and brain dopamine level, thus improving exercise or athletic endurance.

The inventors have found that the composition comprising DHZ of the present invention and the method of the present invention have a positive regulating effect on intestinal flora; can regulate or increase the level of intestinal flora in the subject; increase dopamine in brain; enhance sports and/or athletic and/or exercise performance, including improving endurance, raising heart rate, increasing blood flow, enhancing muscle endurance and increasing muscle strength or motivation.

What is claimed is:

1. A method for regulating or increasing the level of intestinal flora in a subject, wherein the method includes: administering to the subject a composition comprising dehydrozingerone or a salt, acid, ester or derivative thereof; the intestinal flora is Gram-positive bacteria.

2. The method of claim 1, wherein the Gram-positive bacteria are Eubacterium rectale and/or Coprococcus eutactus.

3. The method of claim 1, wherein the method increases dopamine in brain by regulating or increasing the level of intestinal flora in the subject, thereby enhancing exercise performance.

4. The method of claim 3, wherein the enhancing exercise performance includes improving endurance, raising heart rate, increasing blood flow, enhancing muscle endurance and increasing muscle strength or motivation.

5. The method of claim 1, wherein the composition comprising dehydrozingerone or a salt, acid, ester or derivative thereof is formulated into nutritional supplement, food, beverage or animal feed.

6. The method of claim 1, wherein the composition comprising dehydrozingerone or a salt, acid, ester or derivative thereof is prepared into solid preparation or liquid preparation.

7. The method of claim 1, wherein the composition comprising dehydrozingerone or a salt, acid, ester or derivative thereof is in the form of suppository, tablet, pill, granule, powder, film, capsule, beverage, aerosol, spirit, tincture, tonic, liquid suspension or syrup.

8. The method of claim 1, wherein the composition comprising dehydrozingerone or a salt, acid, ester or derivative thereof is administered to the subject at a dosage of 1-1500 mg.

9. The method of claim 1, wherein the subject is human.

10. The method of claim 1, wherein the dopamine level of the subject administered with the composition comprising dehydrozingerone or a salt, acid, ester or derivative thereof is higher than that in the subject without administration.

11. A method for enhancing exercise performance in a subject, wherein the method includes administering to the subject a composition comprising dehydrozingerone or a salt, acid, ester or derivative thereof.

12. The method of claim 11, wherein the method enhances exercise performance in the subject by increasing dopamine in brain.

13. The method of claim 11, wherein the enhancing exercise performance includes improving endurance, raising heart rate, increasing blood flow, enhancing muscle endurance and increasing muscle strength or motivation.

14. The method of claim 11, wherein the composition comprising dehydrozingerone or a salt, acid, ester or derivative thereof is formulated into nutritional supplement, food, beverage or animal feed.

15. The method of claim 11, wherein the composition comprising dehydrozingerone or a salt, acid, ester or derivative thereof is prepared into solid preparation or liquid preparation.

16. The method of claim 11, wherein the composition comprising dehydrozingerone or a salt, acid, ester or derivative thereof is administered in the form of suppository, tablet, pill, granule, powder, film, capsule, beverage, aerosol, spirit, tincture, tonic, liquid suspension, syrup, functional food, beverage or sublingual product.

17. The method of claim 11, wherein the composition comprising dehydrozingerone or a salt, acid, ester or derivative thereof is administered to the subject at a dosage of 1-1500 mg.

18. The method of claim 11, wherein the subject is human.

19. The method of claim 11, wherein the dopamine level of the subject administered with the composition comprising dehydrozingerone or a salt, acid, ester or derivative thereof is higher than that in the subject without administration.

20. The method of claim 11, wherein the exercise endurance of the subject administered with the composition comprising dehydrozingerone or a salt, acid, ester or derivative thereof is higher than that in the subject without administration.
